SEQUENCE LISTING (22 sequences)

SEQ ID NO: 1 - protein, 22 aa, Artificial Sequence
  synthetic peptide
FNAPXXXGIKLXGXXXXHXXXX

SEQ ID NO: 2 - protein, 23 aa, Artificial Sequence
  synthetic peptide
FNAPFDVGIKLSGXXXQQHXXXL

SEQ ID NO: 3 - protein, 24 aa, human
FNAPFDVGIKLSGVQYQQHSQALG

SEQ ID NO: 4 - protein, 23 aa, human
FNAPFDVGIKLSGVQYQQHSQAL

SEQ ID NO: 5 - protein, 24 aa, sheep
FNAPFNIGIKLSGAQSLQHGQTLG

SEQ ID NO: 6 - protein, 23 aa, sheep
FNAPFNIGIKLSGAQSLQHGQTL

SEQ ID NO: 7 - protein, 24 aa, bos
FNAPFNIGIKLAGAQSLQHGQTKG

SEQ ID NO: 8 - protein, 23 aa, bos
FNAPFNIGIKLAGAQSLQHGQTK

SEQ ID NO: 9 - protein, 24 aa, macaca
FNAPFDVGIKLSGVQYQQHSQALG

SEQ ID NO: 10 - protein, 23 aa, macaca
FNAPFDVGIKLSGVQYQQHSQAL

SEQ ID NO: 11 - protein, 24 aa, mouse
FNAPFDVGIKLSGAQYQQHGRALG

SEQ ID NO: 12 - protein, 23 aa, mouse
FNAPFDVGIKLSGAQYQQHGRAL

SEQ ID NO: 13 - protein, 24 aa, rat
FNAPFDVGIKLSGAQYQQHGRALG

SEQ ID NO: 14 - protein, 23 aa, rat
FNAPFDVGIKLSGAQYQQHGRAL

SEQ ID NO: 15 - protein, 24 aa, gerbil
FNAPFDVGIKLSGAQYQQHGRALG

SEQ ID NO: 16 - protein, 23 aa, gerbil
FNAPFDVGIKLSGAQYQQHGRAL

SEQ ID NO: 17 - protein, 24 aa, pig
FNAPCDVGIKLSGAQSDQHGQPLG

SEQ ID NO: 18 - protein, 23 aa, pig
FNAPCDVGIKLSGAQSDQHGQPL

SEQ ID NO: 19 - protein, 24 aa, dog
FNAPFDVGIKLSGPQYHQHGQALG

SEQ ID NO: 20 - protein, 23 aa, dog
FNAPFDVGIKLSGPQYHQHGQAL

SEQ ID NO: 21 - protein, 24 aa, human
FNAPFDVGIKLSGVLYQQHSQALG

SEQ ID NO: 22 - protein, 23 aa, human
FNAPFDVGIKLSGVLYQQHSQAL

(19) United States
(12) Patent Application Publication
(10) Pub. No.: US 2006/0229250 A1
(43) Pub. Date: Oct. 12, 2006

(54) Ghrelin associated peptide and its uses

(76) Inventors: Jian Zhang, Mountain View, CA (US); Orna Avsian-Kretchmer, Fremont, CA (US); Aaron J.W. Hsueh, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/372,888

(22) Filed: Mar. 9, 2006

Related U.S. Application Data
(60) Provisional application No. 60/660,494, filed on Mar. 9, 2005.

(51) Int. Cl. A61K 38/10; C07K 7/08; C07H 21/04; C12P 21/06

(52) U.S. Cl. 514/14; 530/327; 435/69.1; 435/320.1; 435/325; 530/388.1; 536/23.5

(57) ABSTRACT

The invention relates to polypeptides referred to herein as obestatin. Obestatin, analogs and mimetics thereof act in signaling pathways, and are shown to modulate hypertension and other cardiovascular parameters, and gastric emptying.

### INTRODUCTION

Polypeptide hormones and their receptors play important roles in the maintenance of homeostasis in multicellular organisms. Recent sequencing of the genomes of human and several animal models provide an unprecedented opportunity to identify novel polypeptide ligands based on sequence homology among paralogous ligand genes. In addition, a large number of putative G protein-coupled receptors without known ligands have been predicted based on their characteristic seven transmembrane domains. Although the ligands for some of these ‘orphan’ GPCRs have been identified based on biochemical purification and other approaches, the ligands for many of them are still unknown. There is considerable interest for clinical and research purposes in the discovery and development of agents that act on these receptors.

Small synthetic molecules called growth hormone secretagogues (GHSs) stimulate the release of growth hormone from the pituitary. They act through the growth hormone secretagogue receptor, a G protein-coupled receptor. An endogenous ligand specific for GHSR was reported by Kojima et al. (1999) Nature 402: 656-660. The ligand is a peptide of 28 amino acids in which the serine-3 residue is n-octanoylated. The acylated peptide specifically releases growth hormone both in vivo and in vitro, and O-n-octanoylation at serine-3 is essential for the activity. The GH-releasing peptide was termed “ghrelin”. Human ghrelin has substantial sequence identity to rat ghrelin, differing by only 2 amino acids. The occurrence of ghrelin in both rat and human indicates that GH release from the pituitary may be regulated not only by hypothalamic growth hormone-releasing hormone, but also by ghrelin. Ghrelin is expressed in the stomach, apparently by endocrine cells. Ghrelin is also expressed in neurons, e.g. in the hypothalamic arcuate nucleus.

Ghrelin circulates in healthy human blood at a considerable plasma concentration, suggesting that this molecule is produced in and secreted from the stomach, circulating in the bloodstream to act on the pituitary. In addition to the release of growth hormone, ghrelin is involved in the hypothalamic regulation of energy homeostasis. Intracerebroventricular injections of ghrelin strongly stimulated feeding in rats and increased body weight gain. Ghrelin also increased feeding in rats that were genetically deficient in growth hormone. See Nakazato et al. (2001) Nature 409: 194-198; and Tschop et al. (2000) Nature 407: 908-913.

Dixit et al. (2004) J. Clin. Invest. 114: 57-66, 2004 demonstrated that ghrelin and its receptor, GHSR, are expressed in human T lymphocytes and monocytes, where ghrelin acts via GHSR to inhibit specifically the expression of proinflammatory anorectic cytokines such as IL1-beta, IL6, and TNF-alpha. Ghrelin led to a dose-dependent inhibition of leptin-induced cytokine expression, whereas leptin upregulated GHSR expression on human T lymphocytes. Dixit et al. (2004) proposed the existence of a reciprocal regulatory network by which ghrelin and leptin control immune cell activation and inflammation. In a murine model of endotoxemia, Dixit et al. (2004) also showed that ghrelin has potent antiinflammatory effects and attenuates endotoxin-induced anorexia.

### SUMMARY OF THE INVENTION

Obestatin (also referred to as ghrelin associated peptide, or GLAP) and variants thereof are provided. In some embodiments, the peptides are derived by alternative processing of the polypeptide encoded by a ghrelin mRNA transcript. C-terminal amidated forms of obestatin are demonstrated to have a higher activity than the non-amidated forms. Also provided are polynucleotides encoding an obestatin peptide. Preferably such polynucleotides lack sequences encoding ghrelin polypeptides.

The obestatin peptide is found in tissues including the large intestine, small intestine, spleen, cerebral cortex and stomach. Receptors for the peptide are found in tissues including the small intestine, pituitary gland, stomach and ileum. Ghrelin does not compete with obestatin for receptor binding, and can have an opposing effect to ghrelin.

Obestatin peptides act on cells in the gastrointestinal tract, resulting in decreased food intake and weight loss over time. The peptides also decrease blood pressure and heart rate, and affect gastric emptying. In one embodiment of the invention, obestatin peptides find use where it is desirable to regulate blood pressure. In another embodiment, the peptides find use where weight regulation is desirable.

In addition to use as a therapeutic agent, in another embodiment of the invention obestatin peptides are utilized in screening and research methods for the determination of specific analogs, agonists, antagonists mimetics and agents that modulate their production, metabolism, and disposition. Regulatory peptides are ligands for a subgroup of G protein-coupled receptors (GPCRs) and can play important roles in the gastrointestinal, cardiovascular, hypothalamus-pituitary axis, and the central nervous systems.

In one embodiment of the invention, an isolated polypeptide is provided, usually an amidated peptide, and functional fragments, derivatives and homologs thereof. Such polypeptides may be formulated in a pharmaceutical composition comprising a pharmaceutically acceptable carrier.

Polymorphisms in the human obestatin sequence are also provided. In one embodiment of the invention, the genotype of an individual for obestatin is determined.

Other aspects of the invention and their features and advantages will become apparent from the following description and the appended claims.

### BRIEF DESCRIPTION OF THE DRAWINGS

FIGS. 1A-1E. Characterization of endogenous obestatin. (A) Competition of I¹²⁵-obestatin binding to obestatin antibodies by tissue extracts. I¹²⁵-obestatin was incubated with obestatin antibodies with or without different dilutions of tissue extracts and the obestatin standard. pg, picograms of; B, bound; Bo, total bound. (B) Gel permeation chromatography of obestatin in stomach extracts. Stomach tissues from 30 rats were extracted and eluted from a Sep-Pak C-18 column before they were loaded onto a Sephadex G-50 column. The column was calibrated with blue dextran (vo), cytochrome c (cc), and potassium chromate (vt). Peak 1, detected by obestatin antibodies, represents the putative obestatin peptide, and peak 2 represents an obestatin fragment. (C) Ion exchange FPLC analysis of peak 1 fractions monitored by the obestatin immunoassay. (D) Peptide mapping using mass spectrometry and the predicted amino acid sequence of rat obestatin. m, mass; z, charge. (E) Serum levels of ghrelin and obestatin during fasting and refeeding. Adult male rats (n=5 animals per group) were fasted for 2 days. After fasting, some animals were allowed access to food, dextrose solution, or water for 2 hours before the amount of serum hormone was determined using specific radioimmunoassays. Error bars are mean±SEM.

FIGS. 2A-2E. Regulation of gastrointestinal functions by obestatin. (A) Suppression of cumulative food intake after intraperitoneal treatment with obestatin, NA-obestatin, and/or ghrelin. The upper panel shows treatment with different peptides at 1 μmol per kg body weight; the lower panel shows dose response at 5 hours after treatment. Mice injected with urocortin served as positive controls. (B) Suppression of cumulative food intake after intracerebroventricular injection of obestatin. Peptides were injected at 8 nmol per kg body weight. Mice injected with MTII served as positive controls. (C) Treatment with obestatin suppressed body-weight gain. (D) Suppression of gastric emptying activity by obestatin. The upper panel shows treatment with different peptides at 1 μmol per kg body weight; the lower panel shows dose-response relationship at 2 hours after treatment. (E) Treatment with obestatin suppressed the contractile activity of jejunum muscle strips and the stimulatory effect of ghrelin. Representative tracing (upper panel) and percentage of maximal responses (lower panel) are shown. Asterisks indicate P<0.05 versus controls (C). Differences between treatment groups were analyzed using analysis of variance and Student's t-test.

FIG. 3. Circulating leptin and ghrelin levels in rats following long-term treatment with obestatin, ghrelin, and other peptides.

FIG. 4. Ghrelin, but not obestatin, stimulated growth hormone secretion by cultured anterior pituitary cells. Differences between treatment groups were analyzed using ANOVA and Student's t-test.

FIG. 5A-5C. Obestatin binding to target tissues. A) High affinity binding of I₁₂₅-obestatin to plasma membrane preparations of rat jejunum. Crude plasma membrane fractions were incubated with increasing doses of I₁₂₅-obestatin with or without excess of nonlabeled obestatin before determination of specific obestatin binding. B) Hormonal specificity of obestatin binding to rat jejunum. Peptides listed were tested separately. C) Specific binding of I₁₂₅-obestatin to diverse rat tissues.

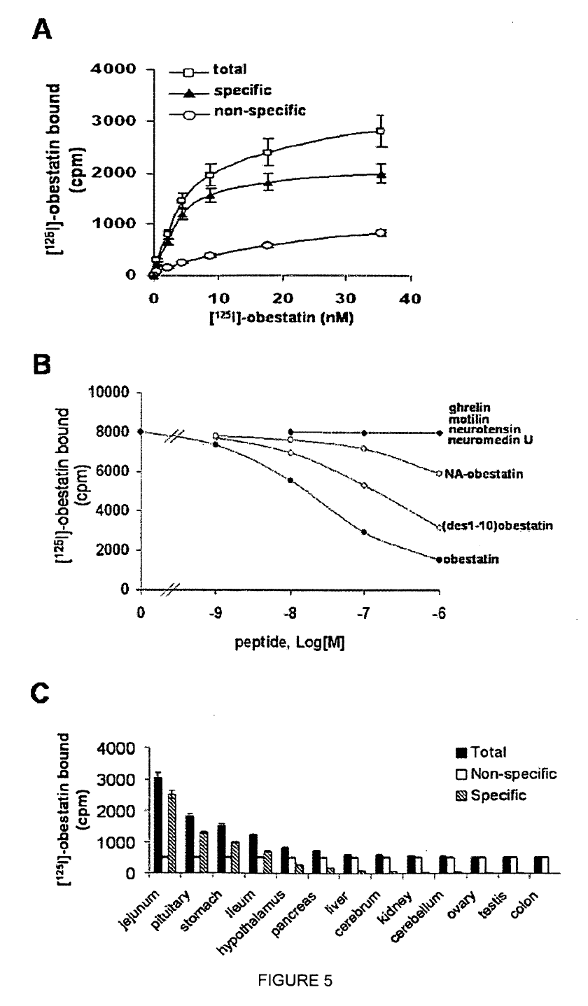

FIG. 6. Binding of obestatin polymorphic variants to jejunal membranes.

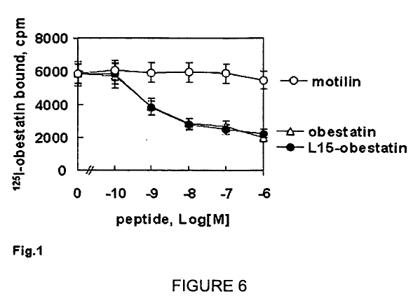

### DETAILED DESCRIPTION OF THE INVENTION AND PREFERRED EMBODIMENTS

The regulatory peptide obestatin is a ligand for the G protein-coupled receptors (GPCRs) that play important roles in gastrointestinal, cardiovascular, hypothalamus-pituitary axis, and the central nervous system. In addition to use as a therapeutic agent, obestatin peptides are utilized in screening and research methods for the determination of specific analogs, agonists, antagonists and mimetics and inhibitors of their production, metabolism and disposition.

In one embodiment of the invention, modulators of obestatin activity are used in the treatment of obesity. In another embodiment, modulators of obestatin activity are used in the modulation of cardiovascular function, including heart rate and blood pressure regulation.

### Obestatin Compositions

Obestatin peptides, which can be used in the methods of the invention, comprise at least about 10 amino acids, usually at least about 12 amino acids, at least about 15 amino acids, at least about 18 amino acids, at least about 21 amino acids, and which may include up to 23 amino acids of a obestatin peptide, or modifications thereof, and may further include fusion polypeptides as known in the art in addition to the provided sequences. A combination of one or more forms may be used. The obestatin sequence may be from any mammalian or avian species, e.g. primate sp., particularly humans; rodents, including mice, rats and hamsters; rabbits; equines, bovines, canines, felines; etc. Of particular interest are the human proteins. Preferably the peptide is modified, where the C terminal glycine residue is replaced with an amide.

In some embodiments, the obestatin peptide has the sequence (SEQ ID NO:1):

F N A P X₁ X₂ X₃ G I K L X₄ G X₅ X₆ X₇ X₈ H X₉ X₁₀ X₁₁ X₁₂ —NH₂
wherein X₂ is phenylalanine or cysteine, preferably phenylalanine (F, C);
X₂ is aspartic acid, asparagine, glutamic acid or glutamine (N, Q, D, E);
X₃ is a hydrophobic residue, e.g. phenylananine, valine, leucine and isoleucine (F, V, L, I);
X₄ is serine, alanine or glycine (A, S, G);
X₅ is alanine, valine, proline, leucine, isoleucine (A, V, P, L, I);
X₆ is glutamine or leucine (Q, L)
X₇ is serine or tyrosine (S, Y);
X₈ is glutamine, leucine, isoleucine, aspartic acid, histidine (Q, L, D, H, I);
X₉ is serine, alanine or glycine (A, S, G);
X₁₀ is glutamine, asparagine, or arginine (Q, N, R);
X₁₁ is alanine, proline, or threonine (A, T, P);
X₁₂ is lysine or leucine (K, L);
   and where the terminal NH₂ indicates the replacement of a glycine residue with an amide.

In a related embodiment, an obestatin peptide of interest has the sequence (SEQ ID NO:2):

F N A P F D V G I K L S G X₁₃ X₁₄ X₁₅ Q Q H X₁₆ X₁₇ X₁₈ L —NH₂
wherein X₁₃ is alanine, valine, proline (A, V, P);
X₁₄ is glutamine or leucine (Q, L);
X₁₅ is serine or tyrosine (S, Y);
X₁₆ is serine or glycine (S, G);
X₁₇ is glutamine or arginine (Q, R);
X₁₈ is alanine or threonine (A, T).

Specific obestatin peptides of interest include the following native obestatin peptides:

| | |
|---|---|
| FNAPFDVGIKLSGVQYQQHSQALG (human) | (SEQ ID NO:3) |
| FNAPFDVGIKLSGVQYQQHSQAL-NH₂ (human) | (SEQ ID NO:4) |
| FNAPFDVGIKLSGVLYQQHSQALG (human) | (SEQ ID NO:21) |
| FNAPFDVGIKLSGVLYQQHSQAL-NH₂ (human) | (SEQ ID NO:22) |
| FNAPFNIGIKLSGAQSLQHGQTLG (sheep) | (SEQ ID NO:5) |
| FNAPFNIGIKLSGAQSLQHGQTL-NH₂ (sheep) | (SEQ ID NO:6) |
| FNAPFNIGIKLAGAQSLQHGQTKG (bos) | (SEQ ID NO:7) |
| FNAPFNIGIKLAGAQSLQHGQTK-NH₂ (bos) | (SEQ ID NO:8) |
| FNAPFDVGIKLSGVQYQQHSQALG (macaca) | (SEQ ID NO:9) |
| FNAPFDVGIKLSGVQYQQHSQAL-NH₂ (macaca) | (SEQ ID NO:10) |
| FNAPFDVGIKLSGAQYQQHGRALG (mouse) | (SEQ ID NO:11) |
| FNAPFDVGIKLSGAQYQQHGRAL-NH₂ (mouse) | (SEQ ID NO:12) |
| FNAPFDVGIKLSGAQYQQHGRALG (rat) | (SEQ ID NO:13) |
| FNAPFDVGIKLSGAQYQQHGRAL-NH₂ (rat) | (SEQ ID NQ:14) |
| FNAPFDVGIKLSGAQYQQHGRALG (gerbil) | (SEQ ID NO:15) |
| FNAPFDVGIKLSGAQYQQHGRAL-NH₂ (gerbil) | (SEQ ID NO:16) |
| FNAPCDVGIKLSGAQSDQHGQPLG (pig) | (SEQ ID NO:17) |
| FNAPCDVGIKLSGAQSDQHGQPL-NH₂ (pig) | (SEQ ID NO:18) |
| FNAPFDVGIKLSGPQYHQHGQALG (dog) | (SEQ ID NO:19) |
| FNAPFDVGIKLSGPQYHQHGQAL-NH₂ (dog) | (SEQ ID NO:20) |

The peptides of the invention have a number of important physiological functions, including modulation of body weight and metabolism, and modulation of cardiovascular activity. Modulators of cardiovascular activity refer to molecules that alter the physiological function of the cardiovascular system, including, without limitation, the blood pressure and heart rate, etc. Modulators of weight affect the intake of food, gastric motility, weight homeostasis, etc. Such modulators include agonists, which enhance, potentiate and/or mimic the activity of a obestatin peptide; and antagonists, which inhibit or decrease the activity of a obestatin peptide.

The sequence of obestatin peptides as described above may be altered in various ways known in the art to generate targeted changes in sequence. The sequence changes may be substitutions, insertions or deletions. Such alterations may be used to alter properties of the protein, by affecting the stability, specificity, etc. Techniques for in vitro mutagenesis of cloned genes are known. Examples of protocols for scanning mutations may be found in Gustin et al., Biotechniques 14:22 (1993); Barany, Gene 37:111-23 (1985); Colicelli et al., Mol Gen Genet 199:537-9 (1985); and Prentki et al., Gene 29:303-13 (1984). Methods for site specific mutagenesis can be found in Sambrook et al., Molecular Cloning: A Laboratory Manual, CSH Press 1989, pp. 15.3-15.108; Weiner et al., Gene 126:35-41 (1993); Sayers et al., Biotechniques 13:592-6 (1992); Jones and Winistorfer, Biotechniques 12:528-30 (1992); Barton et al., Nucleic Acids Res 18:7349-55 (1990); Marotti and Tomich, Gene Anal Tech 6:67-70 (1989); and Zhu Anal Biochem 177:1204 (1989).

The peptides may be joined to a wide variety of other oligopeptides or proteins for a variety of purposes. By providing for expression of the subject peptides, various post-expression modifications may be achieved. For example, by employing the appropriate coding sequences, one may provide farnesylation or prenylation. The peptides may be PEGylated, where the polyethyleneoxy group provides for enhanced lifetime in the blood stream. The peptides may also be combined with other proteins in a fusion protein, typically where the two proteins are not normally joined, such as the Fc of an IgG isotype, which may be complement binding, with a toxin, such as ricin, abrin, diphtheria toxin, or the like, or with specific binding agents that allow targeting to specific moieties on a target cell.

The obestatin for use in the subject methods may be produced from eukaryotic or prokaryotic cells, or may be synthesized in vitro. Where the protein is produced by prokaryotic cells, it may be further processed by unfolding, e.g. heat denaturation, DTT reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are peptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The subject peptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In one embodiment of the invention, the obestatin peptide consists essentially of a polypeptide sequence of at least 23 amino acids in length and having a sequence of an obestatin peptide as described above. By “consisting essentially of” in the context of a polypeptide described herein, it is meant that the polypeptide is composed of the obestatin sequence, which sequence is optionally flanked by one or more amino acid or other residues that do not materially affect the basic characteristic(s) of the polypeptide.

Obestatin polypeptides include those provided herein, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain where the polypeptide is a member of a protein family, or a region associated with a consensus sequence). Variants also include fragments of the polypeptides disclosed herein, for example, biologically active fragments and/or fragments corresponding to functional domains.

The invention includes nucleic acids encoding the peptides of the invention. The nucleic acid sequences encoding the above obestatin polypeptides may be accessed from public databases. Identification of additional obestatins is accomplished by conventional screening methods of DNA libraries or biological samples for DNA sequences having a high degree of similarity to known obestatin sequences. Such polynucleotides preferably lack sequences encoding ghrelin polypeptides, i.e. the polynucleotides of the invention are other than mRNA or corresponding cDNA of naturally occurring ghrelin/obestatin transcripts. Polynucleotides of interest include those that encode a peptide that consists essentially of a polypeptide sequence of at least about 12 amino acids, at least about 15 amino acids, at least about 18 amino acids, at least about 21 amino acids, and which may include up to 23 amino acids having a sequence of a obestatin peptide as described above. Such polynucleotides may be operably joined to control sequences, e.g. for transcriptional start, stop, translation, promoters, etc. Polynucleotides may also include an obestatin coding sequence combined with fusion polypeptide sequences.

Obestatin coding sequences can be generated by methods known in the art, e.g. by in vitro synthesis, recombinant methods, etc. to provide a coding sequence to corresponds to an obestatin polypeptide that can serve as an intermediate in the production of the obestatin peptide. Using the known genetic code, one can produce a suitable coding sequence. Double or single stranded fragments can be obtained from the DNA sequence by chemically synthesizing oligonucleotides in accordance with conventional methods, by restriction enzyme digestion, by PCR amplification, etc.

Obestatin encoding nucleic acids can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

Expression vectors may be used to introduce an obestatin coding sequence into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The nucleic acid may be introduced into tissues or host cells by any number of routes, including viral infection, microinjection, or fusion of vesicles. Jet injection may also be used for intramuscular administration, as described by Furth et al. (1992) Anal Biochem 205:365-368. The DNA may be coated onto gold microparticles, and delivered intradermally by a particle bombardment device, or “gene gun” as described in the literature (see, for example, Tang et al. (1992) Nature 356:152-154), where gold microprojectiles are coated with the obestatin or DNA, then bombarded into skin cells.

### Uses of Obestatin

In light of the pharmacologic activities of obestatin, numerous clinical indications are evident. For example, clinical indications for which an obestatin peptide or variants thereof may find use include treatment of obesity, as a cardioprotective agent, and as a hypotensive agent.

Human obesity is a widespread and serious disorder, affecting a high percentage of the adult population in developed countries. In spite of an association with heart disease, type II diabetes, cancer, and other conditions, few persons are able to permanently achieve significant weight loss. The subject peptides are administered to obese patients for purposes of appetite suppression. Patients may use various criteria for determining obesity. Conveniently, a body mass index (BMI) is calculated, where a person having a BMI greater than 25 is overweight and may considered for treatment with the subject peptides. Obestatins find use in promoting gastric stasis and anorexic behavior without concomitant activation of the ACTH-glucocorticoid axis.

In a related embodiment, the treatment of non-insulin-dependent diabetes mellitus (NIDDM) is closely related to the treatment of obesity. NIDDM is a metabolic disease that affects about 5% to 7% of the population in western countries (and 10% of individuals over age 70). It is characterized by hyperglycemia and often accompanied by a number of other conditions, including hypertension, obesity and lipid disturbances. Patients are generally categorized as diabetic or hyperglycemic by measuring the level of glucose in the blood, either directly or by monitoring the level of glycosylated hemoglobin. Treatment is recommended where fasting glucose levels are greater 140 mg/dl, where bedtime glucose is greater than 160 mg/dl, or where HbA_{1c} is greater than 8%. The level of reduction that is desirable depends on the condition of the patient, and the blood glucose levels at the start of treatment, but generally about a 10 to 40% reduction is blood glucose is desirable, usually about a 25 to 35% reduction.

Hypertension is a disease which, if untreated, strongly predisposes to atherosclerotic cardiovascular disease. It is estimated that as many as 1 in 4 adult Americans have hypertension. Hypertension is approximately twice as common in persons with diabetes as in those without. The prevalence of hypertension increases with age.

Hypertension should not be diagnosed on the basis of a single measurement. Initial elevated readings should be confirmed on at least two subsequent visits over one week or more with average diastolic blood pressure of 90 mmHg or greater or systolic blood pressure of 140 mmHg or greater required for diagnosis of hypertension. Special care is warranted in diagnosing hypertension in persons with diabetes because of greater variability of blood pressure and a much greater likelihood of isolated systolic hypertension. A goal blood pressure of less than 130/85 mmHg is recommended for these patients.

In addition to dietary changes, pharmacological treatment may be required to control high blood pressure. The subject peptides may be administered to reduce arterial blood pressure. In addition, a secondary effect of reducing hypertension is reduction of edema and inflammatory exudate volume.

Pharmaceutical compositions containing obestatin peptides and derivatives therefrom are useful as cardioprotective agents, e.g. to ameliorate ischemic injury or myocardial infarct size consequent to myocardial ischemia. The development of new therapeutic agents capable of limiting the extent of myocardial injury, i.e., the extent of myocardial infarction, following acute myocardial ischemia is a major concern of modern cardiology. There has also been interest in the development of therapies capable of providing additional myocardial protection which could be administered in conjunction with thrombolytic therapy, or alone, since retrospective epidemiological studies have shown that mortality during the first few years following infarction appears to be related to original infarct size.

Myocardial ischemia is the result of an imbalance of myocardial oxygen supply and demand and includes exertional and vasospastic myocardial dysfunction. Exertional ischemia is generally ascribed to the presence of critical atherosclerotic stenosis involving large coronary arteries resulting in a reduction in subendocardial flow. Vasospastic ischemia is associated with a spasm of focal variety, whose onset is not associated with exertion or stress. The spasm is better defined as an abrupt increase in vascular tone.

The compounds of this invention can be normally administered orally or parenterally, in the treatment of patients in need of cardioprotective therapy. The dosage regimen is that which insures maximum therapeutic response until improvement is obtained and thereafter the minimum effective level that gives relief. Thus, in general, the dosages are those that are therapeutically effective in producing a cardioprotective effect, i.e., amelioration of ischemic injury or myocardial infarct size consequent to myocardial ischemia. It is also anticipated that the peptides would be useful as an injectable dosage form, which may be administered in an emergency to a patient suffering from myocardial ischemia, etc.

In one aspect, the invention features a method of beneficially regulating gastrointestinal motility in a subject by administering to said subject a therapeutically effective amount of an obestatin peptide or modulator thereof. In one embodiment, the methods of the present invention are directed to reducing gastric motility. In another embodiment, the invention is directed to methods of delaying gastric emptying. These methods may be used on a subject undergoing a gastrointestinal diagnostic procedure, for example radiological examination or magnetic resonance imaging. Alternatively, these methods may be used to reduce gastric motility in a subject suffering from a gastrointestinal disorder, for example, spasm (which may be associated with acute diverticulitis, a disorder of the biliary tract or a disorder of the Sphincter of Oddi). In another aspect, the invention is directed to a method of treating post-prandial dumping syndrome in a subject by administering to the subject a therapeutically effective amount of an obestatin modulator. In another aspect, the invention is directed to a method of treating post-prandial hyperglycemia by administering to a subject a therapeutically effective amount of an obestatin agonist, e.g. post-prandial hyperglycemia as a consequence of Type 2 diabetes mellitus.

In another aspect, the present invention is directed to a method of treating gastric hypomotility in a subject by administering to the subject a therapeutically effective amount of an obestatin antagonist. These methods may be employed where hypomotility is a consequence of diabetic neuropathy or where hypomotility is a consequence of anorexia nervosa. Hypomotility may also occur as a consequence of achlorhydria or as a consequence of gastric surgery. In another aspect, the invention is directed to a method of accelerating gastric emptying in a subject by administering to the subject a therapeutically effective amount of an obestatin modulator.

### Compound Screening

In another aspect, the invention relates to methods for assaying or screening compounds to determine their activities as modulators of the function of the polypeptides described above. Compound screening may be performed using an in vitro model, a genetically altered cell or animal, or purified protein corresponding to an obestatin or derivative thereof. One can identify ligands, substrates and/or modulators that bind to, modulate or mimic the action of the peptides, including the identification of inhibitors and potentiators of obestatin activity.

Compound screening identifies agents that modulate function of obestatin. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like.

The term “modulator” includes any molecule, e.g. protein or pharmaceutical, with the capability of altering or mimicking the physiological function of an obestatin peptide. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate modulators comprise functional groups necessary for structural interaction with proteins, particularly hydrogen bonding, and typically include at least an amine, carbonyl, hydroxyl or carboxyl group, preferably at least two of the functional chemical groups. The candidate modulators often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate modulators are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to, or interfering in the binding of obestatin to target tissues. The binding assays usually involve contacting obestatin with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots, etc.

The level of expression or activity can be compared to a baseline value. The baseline value can be a value for a control sample or a statistical value that is representative of a control population. Expression or activity levels can also be determined for cells that do not respond to obestatin as a negative control.

Compounds that are initially identified by any of the foregoing screening methods can be further tested to validate the apparent activity. The basic format of such methods involves administering a lead compound identified during an initial screen to an animal that serves as a model for humans and then determining whether the desired biological function is affected. The animal models utilized in validation studies generally are mammals. Specific examples of suitable animals include, but are not limited to, primates, mice, and rats.

Active test agents identified by the screening methods described herein that modulate obestatin activity can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

Once analogs have been prepared, they can be screened using the methods disclosed herein to identify those analogs that exhibit an increased ability to modulate obestatin activity. Such compounds can then be subjected to further analysis to identify those compounds that appear to have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

For receptor binding assays, many of the derived compounds are likely to be antagonists. When compounds capable of binding to the obestatin receptors are found not to exhibit obestatin-like bioactivity, they can be further screened for their antagonistic activities by injecting the compound of interest together with obestatin, and testing their antagonistic properties.

### Pharmaceutical Compositions

Active compounds identified by the screening methods described above and analogs thereof (e.g., pharmaceutically acceptable salts) can serve as the active ingredient in pharmaceutical compositions formulated for the treatment of various disorders as described above. The active ingredient is present in a therapeutically effective amount, i.e., an amount sufficient when administered to substantially modulate the effect of the targeted protein or polypeptide to treat a disease or medical condition mediated thereby.

The compositions can also include various other agents to enhance delivery and efficacy, e.g. to enhance delivery and stability of the active ingredients.

Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

When the pharmaceutical composition includes a polypeptide as the active ingredient, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lies within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, or intracranial method.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which are composed of the packaged active ingredient with a suppository base. Suitable suppository bases include natural or synthetic triglycerides or paraffin hydrocarbons. In addition, it is also possible to use gelatin rectal capsules, which are composed of a combination of the packaged active ingredient with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are preferably sterile. To the extent that a given compound must be synthesized prior to use, the resulting product is preferably substantially free of any potentially toxic agents, such as any endotoxins, which may be present during the synthesis or purification process. Compositions for parental administration are also preferably sterile, substantially isotonic and made under GMP conditions.

### Antibodies Specific for Obestatin Polypeptides

The present invention further provides antibodies specific for obestatin polypeptides, e.g. any one of the variants or polypeptides described above. Such antibodies are useful, for example, in methods of detecting the presence of obestatin in a biological sample, and in methods of isolating obestatin from a biological sample. Antibodies may also be useful as antagonists of obestatin activity.

The obestatin polypeptides of the invention are useful for the production of antibodies, where short fragments provide for antibodies specific for the particular polypeptide, and larger fragments or the entire protein allow for the production of antibodies over the surface of the polypeptide. As used herein, the term “antibodies” includes antibodies of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme that generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions indicates that a given antibody binds to a given antigen, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for an obestatin polypeptide, particularly a human obestatin polypeptide.

Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For a more detailed description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

### Modulation of Obestatin Expression

Obestatin genes, gene fragments, or the encoded protein or protein fragments are useful in gene therapy to treat disorders associated with obestatin defects. Antisense obestatin sequences may be administered to inhibit expression. Other inhibitors or modulators of expression are identified by screening for biological activity in a obestatin-based binding assay.

Expression vectors may be used to introduce an obestatin coding sequence into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more preferably for a period of at least about several days to several weeks.

The gene may be introduced into tissues or host cells by any number of routes, including viral infection, microinjection, or fusion of vesicles. Jet injection may also be used for intramuscular administration, as described by Furth et al. (1992) Anal Biochem 205:365-368. The DNA may be coated onto gold microparticles, and delivered intradermally by a particle bombardment device, or “gene gun” as described in the literature (see, for example, Tang et al. (1992) Nature 356:152-154), where gold microprojectiles are coated with the DNA, then bombarded into skin cells.

Antisense molecules can be used to down-regulate expression of obestatin in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, preferably at least about 12, more preferably at least about 20 nucleotides in length, and not more than about 100, preferably not more than about 50, more preferably not more than about 35 nucleotides in length, where the length is governed by efficiency of inhibition, specificity, including absence of cross-reactivity, and the like.

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in vitro or in an animal model. A combination of sequences may also be used, where several regions of the mRNA sequence are selected for antisense complementation.

Antisense oligonucleotides may be chemically synthesized by methods known in the art. Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

### Diagnostic Uses

DNA-based reagents derived from the sequence of obestatin; e.g. PCR primers, oligonucleotide or cDNA probes, as well as antibodies against obestatin, are used to screen patient samples, e.g. biopsy-derived tissues, blood samples, and the like, for altered expression of obestatin mRNA or proteins. DNA-based reagents are also designed for evaluation of chromosomal loci implicated in certain diseases e.g. for use in loss-of-heterozygosity (LOH) studies, or design of primers based on obestatin coding sequence.

Genotyping of polymorphic obestatin alleles may be used in the evaluation of an individual, e.g. whether an individual will respond well to a particular therapeutic regimen. In one embodiment of the invention, an individual is screened for the presence of an L15 polymorphism (e.g. as shown in SEQ ID NO:24). Diagnosis of conditions associated with polymorphisms may be performed by protein, DNA or RNA sequence and/or hybridization analysis of any convenient sample from a patient, e.g. biopsy material, blood sample, scrapings from cheek, etc. Individuals are screened by analyzing their DNA or mRNA for the presence of a polymorphism.

The polynucleotides of the invention can be used to detect differences in expression levels between two samples. A difference between the protein levels, or the mRNA in the two tissues that are compared, for example, in molecular weight, amino acid or nucleotide sequence, or relative abundance, indicates a change in the gene, or a gene, which regulates it, in the tissue of the human that was suspected of being diseased.

The subject nucleic acid and/or polypeptide compositions may be used to analyze a patient sample for the presence of polymorphisms associated with a disease state or genetic predisposition to a disease state. Biochemical studies may be performed to determine whether a sequence polymorphism in an obestatin coding region or control regions is associated with disease, such as stress related disorders, e.g. anxiety disorders. Disease associated polymorphisms may include deletion or truncation of the gene, mutations that alter expression level, that affect the binding activity of the protein, the kinase activity domain, and the like.

Changes in the promoter or enhancer sequence that may affect expression levels of obestatin can be compared to expression levels of the normal allele by various methods known in the art. Methods for determining promoter or enhancer strength include quantitation of the expressed natural protein; insertion of the variant control element into a vector with a reporter gene such as β-galactosidase, luciferase, and chloramphenicol acetyltransferase which provides for convenient quantitation; and the like.

A number of methods are available for analyzing nucleic acids for the presence of a specific sequence, e.g. a disease associated polymorphism. Where large amounts of DNA are available, genomic DNA is used directly. Alternatively, the region of interest is cloned into a suitable vector and grown in sufficient quantity for analysis. Cells that express obestatin may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook et al., Molecular Cloning: A Laboratory Manual, CSH Press 1989, pp. 14.2-14.33.

A detectable label may be included in an amplification reaction. Suitable labels include fluorochromes, e.g. fluorescein isothiocyanate (FITC), rhodamine, Texas Red, phycoerythrin, allophycocyanin,6-carboxyfluorescein(6-FAM),2,7-dimethoxy4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²P, ³⁵S, and ³H. The label may be a two stage system, where the amplified DNA is conjugated to biotin, haptens, and the like having a high affinity binding partner, e.g. avidin and specific antibodies., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g., amplified or cloned fragment, is analyzed by one of a number of methods known in the art. The nucleic acid may be sequenced by dideoxy or other methods, and the sequence of bases compared to a wild-type obestatin sequence. Hybridization with the variant sequence may also be used to determine its presence, by Southern blots, dot blots, and the like. The hybridization pattern of a control and variant sequence to an array of oligonucleotide probes immobilized on an array may also be used as a means of detecting the presence of variant sequences. Single strand conformational polymorphism (SSCP) analysis, denaturing gradient gel electrophoresis (DGGE), and heteroduplex analysis in gel matrices may be used to detect conformational changes created by DNA sequence variation as alterations in electrophoretic mobility. Alternatively, where a polymorphism creates or destroys a recognition site for a restriction endonuclease, the sample is digested with that endonuclease, and the products size fractionated to determine whether the fragment was digested. Fractionation is performed by gel or capillary electrophoresis, preferably acrylamide or agarose gels.

Screening for mutations in obestatin may be based on the functional or antigenic characteristics of the protein. Protein truncation assays are useful in detecting deletions that may affect the biological activity of the protein. Various immunoassays designed to detect polymorphisms in obestatin proteins may be used in screening. Where many diverse genetic mutations lead to a particular disease phenotype, functional protein assays have proven to be effective screening tools. The activity of the encoded obestatin peptide in binding assays may be determined by comparison with the wild-type protein. Proteins may also be screened for the presence of post-translational modification of the obestatin peptides, e.g. under pathological conditions, including proteolytic fragments, amidation, and acetylation.

Antibodies specific for obestatin may be used in staining or in immunoassays. Samples, as used herein, include biological fluids such as blood, cerebrospinal fluid, dialysis fluid and the like; organ or tissue culture derived fluids; and fluids extracted from physiological tissues. Also included are derivatives and fractions of such fluids. The cells may be dissociated, in the case of solid tissues, or tissue sections may be analyzed. Alternatively a lysate of the cells may be prepared.

Diagnosis may be performed by a number of methods to determine the absence or presence or altered amounts of normal or abnormal obestatin in patient cells. For example, detection may utilize staining of cells or histological sections, performed in accordance with conventional methods. Cells are permeabilized to stain cytoplasmic molecules. The antibodies of interest are added to the cell sample, and incubated for a period of time sufficient to allow binding to the epitope, preferably at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g. fluorescein rhodamine, Texas red, and the like. Final detection uses a substrate that undergoes a color change in the presence of the peroxidase. The absence or presence of antibody binding may be determined by various methods, including flow cytometry of dissociated cells, microscopy, radiography, scintillation and counting.

In some embodiments, the methods are adapted for use in vivo. In these embodiments, a detectably-labeled moiety, e.g., an antibody, which is specific for obestatin is administered to an individual (e.g., by injection), and labeled cells are located using standard imaging techniques, including, but not limited to, magnetic resonance imaging, computed tomography scanning, and the like.

Diagnostic screening may also be performed for polymorphisms that are genetically linked to a disease predisposition, preferably through the use of microsatellite markers or single nucleotide polymorphisms. The microsatellite polymorphism itself is in many cases not phenotypically expressed, but is linked to sequences that result in a disease predisposition. However, in some cases the microsatellite sequence itself may affect gene expression. Microsatellite linkage analysis may be performed alone, or in combination with direct detection of polymorphisms, as described above. The use of microsatellite markers for genotyping is well known. For examples, see Mansfield et al. (1994) Genomics 24:225-233; Ziegle et al. (1992) Genomics 14:1026-1031; Dib et al., supra.

The detection methods can be provided as part of a kit. Thus, the invention further provides kits for detecting the presence of an mRNA encoding obestatin, and/or a polypeptide encoded thereby, in a biological sample. Procedures using these kits may be performed by clinical laboratories, experimental laboratories, medical practitioners, or private individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

### Genetically Altered Cell of Animal Models for Obestatin Function

The subject nucleic acids can be used to generate transgenic animals or site specific gene modifications in cell lines. Transgenic animals may be made through homologous recombination, where the normal obestatin locus is altered. Alternatively, a nucleic acid construct is randomly integrated into the genome. Vectors for stable integration include plasmids, retroviruses and other animal viruses, YACs, and the like.

The modified cells or animals are useful in the study of obestatin function and regulation. For example, a series of small deletions and/or substitutions may be made in the obestatin coding sequence to determine the role of different residues in receptor binding or signal transduction. In one embodiment, obestatin is used to construct transgenic animal models for disorders where expression of obestatin is specifically altered, i.e. reduced, increased, or absent. Specific preferred constructs include anti-sense obestatin which will block obestatin expression and expression of dominant negative obestatin mutations. A detectable marker, such as lac Z, may be introduced into the obestatin locus, where up-regulation of obestatin expression will result in an easily detected change in phenotype.

One may also provide for expression of the obestatin gene or variants thereof in cells or tissues where it is not normally expressed or at abnormal times of development. By providing expression of obestatin protein in cells in which it is not normally produced, one can induce changes in cell behavior, e.g. in the control of cell growth and tumorigenesis.

DNA constructs for homologous recombination will comprise at least a portion of the obestatin coding sequence with the desired genetic modification, and will include regions of homology to the target locus. The regions of homology may include coding regions, or may utilize intron and/or genomic sequence. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al. (1990) Methods in Enzymology 185:527-537.

For embryonic stem (ES) cells, an ES cell line may be employed, or embryonic cells may be obtained freshly from a host, e.g. mouse, rat, or guinea pig. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in culture. The transgenic animals may be any non-human mammal, such as laboratory animals and domestic animals. The transgenic animals may be used in functional studies, drug screening, and the like to determine the effect of a candidate drug on stress responses.

### Experimental

The following examples are put forth for illustrative purposes, and are not intended to limit the scope of what the inventors regard as their invention. Unless indicated otherwise, parts are parts by weight, molecular weight is weight average molecular weight, temperature is in degrees Celsius, and pressure is at or near atmospheric.

### EXAMPLE 1

The increasing prevalence of obesity is a global problem. Body weight is regulated in part by peptide hormones produced in the brain or gut or both. Earlier studies on synthetic and peptidyl growth hormone (GH) secretagogues led to the identification of a specific G protein-coupled receptor (GPCR), the GH secretagogue receptor (GHSR), and subsequently to the discovery of its endogenous ligand, ghrelin, a gut-derived circulating hormone that stimulates food intake.

Human ghrelin, a 28-amino acid peptide, is derived by posttranslational cleavage from a prepropeptide of 117 residues. We identified a ghrelin-associated peptide in 11 mammalian species. The coding sequence, within pro-ghrelin, is flanked by convertase cleavage sites. This region encodes a putative 23-amino acid peptide, with a flanking conserved glycine residue at the C terminus that is amidated. We named this ghrelin-associated peptide obestatin.

Materials and Methods

Reagents. Human obestatin, nonamidated obestatin (NA-obestatin), and (des1-10) obestatin were synthesized by GL Biochem Ltd. (Shanghai, China) and purified by reverse phase high performance liquid chromatography. The peptide sequences were verified by amino acid analysis and mass spectrometry. Ghrelin, motilin, neurotensin, neuromedin U, and MTII were purchased from Phoenix Pharmaceuticals (Belmont, Calif.). The rabbit polyclonal antibodies against synthetic human obestatin peptide were produced by Quality Controlled Biochemicals (Hopkinton, Mass.).

Immunoassays for obestatin, ghrelin, and leptin. Tissue extracts and sera were used for the measurement of obestatin and ghrelin immunoreactivities. Immuno-obestatin was determined using rabbit polyclonal antibodies at a final dilution of 1:6,000. The reaction mixture consisted of 100 μl of test samples or standards together with 100 μl of the antiserum. Samples were incubated for 24 hours at 4° C., 100 μl of tracer (10,000-15,000 cpm) was added, and the samples further incubated for another 24 hours at 4° C. Free and bound obestatin were separated by the solid phase second antibody method using donkey anti-rabbit IgG (Phoenix Pharmaceuticals) before counting in a γ-spectrometer (LKB, Uppsala, Sweden). Total (n-octanoyl and des-acyl) ghrelin immunoreactivity was determined using a specific radioimmunoassay (Phoenix Pharmaceuticals). No cross-reactivity was found between the obestatin and ghrelin radioimmunoassays.

Circulating ghrelin and obestatin levels were measured in adult male Sprague-Dawley rats before and after fasting for 48 hours, or fasting followed by free access to food or drinking water containing 50% dextrose for 2 hours. Following long term treatment with obestatin, serum leptin levels were determined using an enzyme-linked immunosorbent assay (Phoenix Pharmaceuticals).

Purification of obestatin. Stomach preparations (67 g) from 30 rats were minced and boiled for 5 min. in 5 volumes of water to inactivate intrinsic proteases. Before homogenization with a Polytron mixer, the solution was adjusted to 1 M acetic acid and 20 mM HCl. After centrifugation at 225,000 g for 30 min., the supernatant was concentrated to 100 ml using an evaporator before precipitation under 66% acetone. After removal of the precipitates, the volume of the supernatant was reduced by acetone evaporation before loading onto a 10-g cartridge of Sep-Pak C18 (Waters, Milford, Mass.) pre-equilibrated with 0.1% trifluoroacetic acid (TFA). The Sep-Pak cartridge was washed with 10% acetonitrile/0.1% TFA, and then eluted with 60% acetonitrile/0.1% TFA. Peptides in the eluate were lyophilized, dissolved in 1 M acetic acid, and fractionated using a Sephadex G-50 gel filtration column. A portion of each fraction was used for obestatin and ghrelin radioimmunoassays. Fractions containing immuno-obestatin were further separated by ion-exchange FPLC on a UNO Q1 column (BioRad) at pH 8.1. After identification of the peak containing immuno-obestatin, the samples were subjected to mass spectrometry and de novo N-terminal sequencing at Pan Facility (Stanford University, Calif.).

Labeling of obestatin and receptor binding. Iodination of obestatin was performed using the lodogen (Pierce, Upland, Ind.) procedure. Mixtures of the peptide (20 μg) and 1 mCi [125I] Nal were transferred to precoated lodogen vials and incubated for 4 min. The 125I-labeled peptide was applied to a Sep-Pak C18 cartridge (Waters) before elution with 60% acetonitrile/0.1% TFA. For radioligand binding assays, rat jejunum or other tissues were washed with buffer A (20 mM Hepes, 5 mM EDTA, 1 mM dithiothreitol (DTT), 10 μM amidinophenylmethanesulfonyl fluoride, 5 mg/L leupeptin, 100 mM KCl, pH 7.5), cut into small pieces, and homogenized using a motorized homogenizer. The homogenates were centrifuged at 1,000 g for 5 min. and the supernatant was centrifuged at 300,000 g for 1 hour at 2° C. The pellets (crude membrane fractions) were resuspended with buffer A without KCl, quickly frozen under liquid nitrogen, and stored at −80° C. until use.

Tissue homogenates were incubated in 100 μl of phosphate buffered saline containing 0.1% bovine serum albumin for 18 hours at room temperature with varying concentrations of 125I-obestatin in the presence or absence of unlabeled obestatin at 1,000-fold excess. After incubation, the tubes were centrifuged for 10 min. at 10,000 g, and pellets were washed twice in ice-cold PBS before quantitation of radioactivity with a γ-spectrophotometer. Specific binding was calculated by subtracting nonspecific binding from total binding. For displacement curves, a fixed concentration of ¹²⁵I-obestatin was incubated with or without increasing concentrations of obestatin or other peptides.

Analysis of gastrointestinal functions. Eight-week-old C57BL6 male mice were housed individually in a regulated environment. Before intraperitoneal treatment with different peptides, mice were deprived of food for 16 hours with free access to water. Food intake was measured by placing preweighed pellets in the cage and weighing uneaten pellets at 5 hours after treatment. For intracerebroventricular injection of different peptides, mice were deprived of food for 16 hours. Free-hand injections were performed at 2 hours after light onset and food intake monitored for 5 hours. To estimate gastric emptying responses, mice deprived of food for 16 hours were given food pellets for 90 min. before injection of different hormones or saline. After treatment, mice were deprived of food again and sacrificed 0.5, 1, and 2 hours later. The stomach was excised at the pylorus and cardia before weighing. Gastric emptying was calculated by subtracting the stomach weight of treated mice from those sacrificed at the time of peptide injection.

Isometric force measurements. Strips of jejunal muscle (˜1 cm in length) were cut along the longitudinal axis of the circular muscle layer and the mucosa was removed to minimize contamination with endogenous peptides. Muscle strips were mounted to a TIS8105R (Kent Scientific Corporation, Torrington, Conn.) isometric strain gauge and immersed in a 5 ml organ bath maintained at 37° C. with oxygenated KRB [Krebs-Ringer phosphate buffer, consisting of 50 mM HEPES, 100 mM NaCl, 5 mM KCl, and 1 mM each of MgCl2, NaH2PO4, and CaCl2] as previously described. A resting force of 1.0 g was applied to intestinal muscles and a 1.5 hours equilibration period was allowed before testing of different peptides during a 5 min. period. After each test, muscle strips were washed with fresh KRB for 20 min. to allow full recovery of basal contractile activities. Maximal contraction was evaluated at the beginning and end of each experiment after treatment with 10 μM of acetylcholine chloride. Contractility data were digitized and stored in a computer using DAS Wizard™ (Measurement Computing, Middleboro, Mass.). The magnitude of contractile strength is expressed as percent of maximal contraction induced by acetylcholine.

Pituitary cell cultures. Anterior pituitaries were removed from adult male Sprague-Dawley rats and dispersed with 0.3% collagenase, 0.1% hyaluronidase and DNase I (10 μg/ml). The cell suspension was centrifuged at 300 g for 5 min. and cells were washed twice before incubation in Dulbecco minimal essential medium (DMEM) containing 0.1% bovine serum albumin (BSA), penicillin (100 units/ml), streptomycin (100 μg/ml), fungizone (2.5 μg/ml), and 10% fetal calf serum. Cells were seeded onto poly-lysine-coated 24-well plates at a density of ˜3×10⁵ cells/well. Cultures were kept in a humidified atmosphere of 5% CO₂ and 95% air at 37° C. After a 72 h-incubation, cells were washed with DMEM containing 0.1% BSA, then incubated for 1 hour in serum-free medium with ghrelin or obestatin. Media was removed for growth hormone measurement by an enzyme-linked immunosorbent assay.

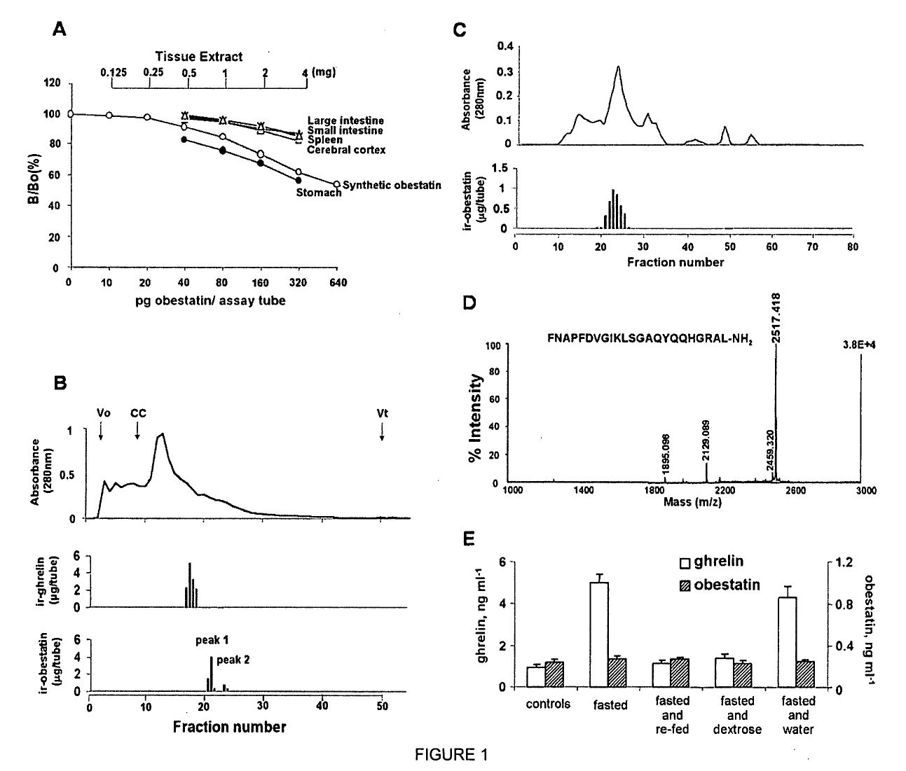

Characterization of endogenous obestatin. To detect endogenous obestatin, we prepared a synthetic obestatin peptide and performed radioimmunoassays on rat-tissue extracts with obestatin-specific antibodies. As shown in FIG. 1A, the stomach extract displaced I¹²⁵-obestatin binding to the obestatin antibodies. Obestatin-like activities from stomach extracts were purified. Immunoreactive (ir) obestatin was eluted in a Sephadex G-50 gel permeation column (Amersham Biosciences, Piscataway, N.J.) with estimated sizes of 2.6 and 1.5 kilo-daltons (kD), distinct from the elution position of mature ghrelin (FIG. 1B). We subjected peak 1 (2.6 kD) fractions to ion-exchange fast protein liquid chromatography (FPLC). A single peak of ir obestatin was eluted (FIG. 1C) and shown by mass spectrometry and Edman sequencing to contain a peptide with a molecular mass of 2516.3 (FIG. 1D) and with a sequence of FNAPFDVGIKLSGAQYQQHG-XX. Combined with molecular-weight determination, the full sequence of the purified peptide was predicted to be FNAPFDVGIKLSGAQYQQHGRALNH₂, consistent with the obestatin sequence deduced from rat ghrelin cDNA. In addition, mass spectrometric analyses suggested that peak 2 (1.5 kD) represented the last 13 residues of amidated obestatin, indicating further processing.

To investigate differential secretion of ghrelin and obestatin in vivo, we fasted adult male rats for 48 hours before refeeding. Consistent with earlier findings, fasting led to a major increase in serum ghrelin levels, whereas subsequent refeeding for 2 hours by allowing animals free access to food or drinking water containing dextrose decreased circulating ghrelin (FIG. 1E). In contrast, serum levels of obestatin determined by a radioimmunoassay were constant in the different treatment groups.

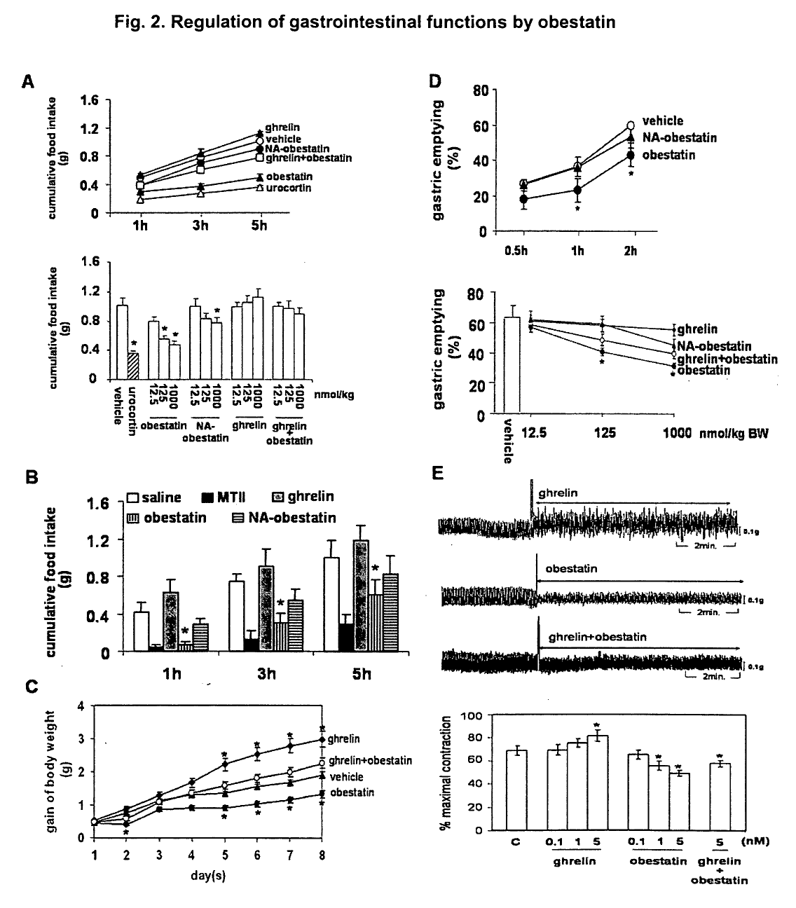

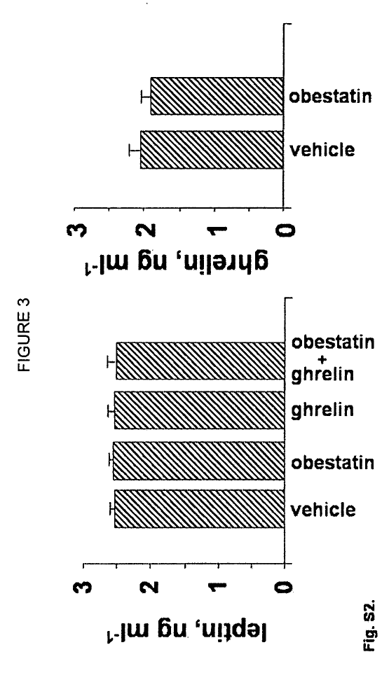

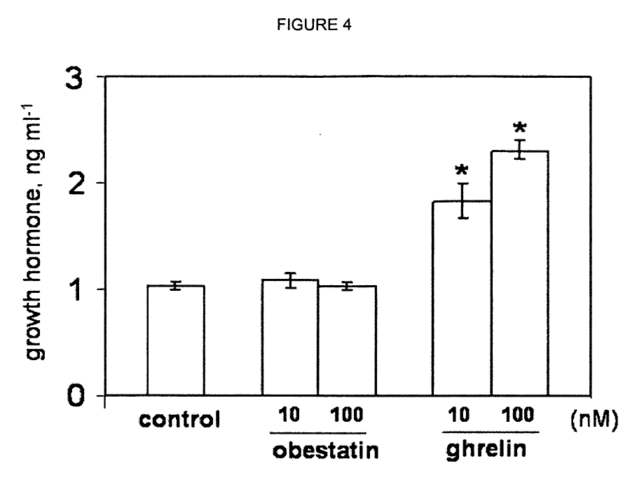

Obestatin suppression of food intake and gastrointestinal functions. We next synthesized amidated human obestatin and tested its effect on food intake in adult male mice. Intraperitoneal injection of obestatin suppressed food intake in a time- and dose-dependent manner (FIG. 2A). Intracerebroventricular treatment with obestatin also decreased food intake (FIG. 2B), similar to the anorexigenic effect of the synthetic melanocortin agonists MTII. In contrast, treatment with the nonamidated obestatin (NA-obestatin) was less effective. We also investigated the effect of obestatin, ghrelin, or vehicle alone on body weight in adult male rats. Treatment with ghrelin (1 μmol per kg body weight, three times daily) increased body weight, whereas the same dose of obestatin suppressed body-weight gain (FIG. 2C). Serum leptin levels were not affected after treatment with either obestatin or ghrelin (FIG. 3), suggesting minimal modulation of body-fat content. Furthermore, treatment with obestatin led to a sustained suppression of gastric emptying activity (FIG. 2D). In vitro, isometric force measurement demonstrated that obestatin treatment decreased the contractile activity of jejunum muscle strips and antagonized the stimulatory effect of ghrelin (FIG. 2E). The observed inhibition of jejunal contraction may trigger an afferent vagus signal to induce a central satiety response. Unlike ghrelin, obestatin did not increase GH secretion by cultured rat pituitary cells (FIG. 4).

Ghrelin is implicated in meal initiation and body-weight regulation. Chronic ghrelin administration increases food intake and decreases energy expenditure, thus causing weight gain. In contrast to ghrelin, which causes hyperphagia and obesity in rats, obestatin appears to act as an anorexic hormone by decreasing food intake, gastric emptying activities, jejunal motility, and body-weight gain. Mutant mice with a deletion of the ghrelin gene did not show impaired growth or appetite, most likely because these animals lacked both orexigenic ghrelin and anorexic obestatin. Indeed, transgenic mice bearing the preproghrelin gene under the control of the chicken β-actin promoter produced high levels of inactive des-acyl ghrelin but exhibited lower body weights, most likely due to excessive obestatin biosynthesis.

The finding that two peptide hormones derived from the same proprotein act through distinct receptors and exert opposing physiological actions highlights the importance of posttranslational regulatory mechanisms. Thus, monitoring of ghrelin transcript levels does not accurately reflect the secretion of these two polypeptides. After removal of the signal peptides from prepropeptides, convertases cleave prohormones at mono- or dibasic residues. In processed peptides with a C-terminal glycine, the residue is further amidated.

In addition to roles in meal initiation, weight regulation, and gastrointestinal activity, ghrelin also regulates the pituitary hormone axis, carbohydrate metabolism, and various functions of the heart, kidney, pancreas, adipose tissues, and gonads. Because ghrelin mRNA was found in almost all human tissues analyzed, the identification of obestatin derived from the same gene product as ghrelin provides a basis for future elucidation of the differential posttranslational processing and modification of these two peptides. A better understanding of the roles of ghrelin and obestatin in the intricate balance of energy homeostasis and body-weight control may be essential for the successful treatment of obesity.

### EXAMPLE 2

Polymorphic forms of obestatin and GPR39 exhibit differential ligand binding. Validation of the polymorphic forms of both obestatin and GPR39 (Genbank accession number AF034633 have been obtained. Obestatin has a 269T→A polymorphism that changes Gln15 to Leu in the mature peptide whereas GPR39 has a 149G→A polymorphism that changes Ala 50 to Val in the first transmembrane region.

The estimated SNP heterozygosity for obestatin is 0.069, with major and minor allele frequencies of 0.964 and 0.036, respectively, taken from a study of 269 people of four populations. In contrast, the estimated SNP heterozygosity for GPR39 is 0.413, with major and minor allele frequencies of 0.709 and 0.291, respectively, from a study of 331 people of seven populations.

The L15-obestatin variant has been synthesized, and site-directed mutagenesis was performed to generate the A50V GPR39 variant. As shown in FIG. 10, ligand-binding assays using iodinated wild type obestatin and rat jejunal membranes indicated that the L15-obestatin variant competed for labeled obestatin binding with a potency similar to the wild type obestatin. Saturation binding of iodinated wild type obestatin to CHO cells was performed with transfected with wild type GPR39 or the 50V-GPR39 variant.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. Accordingly, it should be understood that the scope of the invention is not limited by this detailed description, but by the appended claims as properly construed under principles of patent law.

What is claimed is:

1. An isolated polypeptide comprising the sequence:
F N A P X₁ X₂ X₃ G I K L X₄ G X₅ X₆ X₇ X₈ H X₉ X₁₀ X₁₁ X₁₂ —NH₂
wherein X₁ is phenylalanine or cysteine, preferably phenylalanine (F, C);
X₂ is aspartic acid, asparagine, glutamic acid or glutamine (N, Q, D, E);
X₃ is a hydrophobic residue, e.g. phenylananine, valine, leucine and isoleucine (F, V, L, I);
X₄ is serine, alanine or glycine (A, S, G);
X₅ is alanine, valine, proline, leucine, isoleucine (A, V, P, L, I);
X₆ is glutamine or leucine (Q, L)
X₇ is serine or tyrosine (S, Y);
X₈ is glutamine, leucine, isoleucine, aspartic acid, histidine (Q, L, D, H, I);
X₉ is serine, alanine or glycine (A, S, G);
X₁₀ is glutamine, asparagine, or arginine (Q, N, R);
X₁₁ is alanine, proline, or threonine (A, T, P);
X₁₂ is lysine or leucine (K, L).

2. An isolated polypeptide comprising the sequence:
F N A P F D V G I K L S G X₁₃ X₁₄ X₁₅ Q Q H X₁₆ X₁₇ X₁₈ L —NH₂
wherein X₁₃ is alanine, valine, proline (A, V, P);
X₁₄ is glutamine or leucine (Q, L);
X₁₅ is serine or tyrosine (S, Y);
X₁₆ is serine or glycine (S, G);
X₁₇ is glutamine or arginine (Q, R);
X₁₈ is alanine or threonine (A, T).

3. An isolated polypeptide comprising a sequence seq forth in the group consisting of SEQ ID NO:3 to SEQ ID NO:22.

4. An isolated polypeptide consisting essentially of a sequence set forth in claim 1.

5. An isolated polypeptide comprising at least 6 contiguous amino acids of the sequence set forth in claim 1.

6. An isolated polynucleotide encoding a polypeptide as set forth in claim 4.

7. A vector comprising an isolated nucleic acid molecule as defined in claim 6, operably linked with a promoter sequence.

8. A host cell transformed with the vector of claim 7.

9. A pharmaceutical composition comprising: a therapeutically effective amount of an obestatin peptide; and a pharmaceutically acceptable carrier.

10. A method of modulating gastrointestinal motility, the method comprising administering to an individual a pharmaceutical composition as set forth in claim 9.

11. A method of modulating hypertension, the method comprising administering to an individual a pharmaceutical composition as set forth in claim 9.

12. A method of modulating weight, the method comprising administering to an individual a pharmaceutical composition as set forth in claim 9.

13. An antibody that specifically recognizes an obestatin peptide.

14. A method of screening compounds to identify biologically active agents that modulate obestatin function, the method comprising:
combining a compound with:
(a) a mammalian obestatin peptide;
(b) a cell comprising a nucleic acid encoding a mammalian obestatin peptide; or
(c) a non-human transgenic animal model for obestatin gene function comprising (i) a knockout of an obestatin gene or (ii) an exogenous and stably transmitted mammalian obestatin gene sequence; and
determining the effect of said compound on obestatin function.
